(19) United States
(12) Patent Application Publication
(10) Pub. No.: US 2004/0153039 A1
(43) Pub. Date: Aug. 5, 2004

(54) Disposable butterfly needle sheath

(76) Inventor: Hsien Tsung Wang, Taipei (TW)

Correspondence Address: Hsien Tsung Wang, 4FL. No. 27, Lane 160, Hsin Sheng S. Rd., Sec. 1, Taipei, 100, TW

(21) Appl. No.: 10/734,270

(22) Filed: Dec. 15, 2003

(30) Foreign Application Priority Data
Jan. 30, 2003 (TW) 092102168

(51) Int. Cl. A61M 5/00

(52) U.S. Cl. 604/263; 604/165.03

(57) ABSTRACT

A needle sheath assembly comprises a butterfly needle coupled to an IV tubing, and a plastic sheath comprising two rear side slits, two pairs of upper and lower snapping members, a latched member in front of the slits, a slot extended forward from the latched member. The needle is concealed in the sheath. The upper and lower snapping members secured together. In use push the wings forward along the slot until the needle projects from the aperture. After use pull the wings rearward until the needle has concealed in the sheath and a joint of the wings has moved in the slits prior to pushing the wings forward again until being stopped by the latched member. This can eliminate a possibility of accidentally pricking a medical worker after use. Also, criminals are prevented from selling needle sheath assemblies to hospitals again by simply cleaning or even disinfecting the used needles.

### BACKGROUND OF THE INVENTION

1. Field of the Invention

The present invention relates to hypodermic needles and more particularly to a butterfly needle sheath adapted to couple to an intravenous (IV) infusion set. After its use, a needle in the sheath can be concealed and locked prior to throwing away the needle sheath for safety reasons.

2. Description of Related Art

A wide variety of butterfly needle sheaths are commercially available. For example, Taiwanese Patent Published No. 221,556 entitled “Butterfly Needle Sheath Assembly With Improved Safety Characteristic” disclosed a plastic sheath, a V-shaped groove in a forward end of the sheath, the groove having an arcuate mouth, one or two (e.g., left and right ones) intermediate, elongated gaps, a rearward elongated rectangular slot, and two slanted portions in a forward end of the slot so that wings of a butterfly needle mechanism are locked in the slot when the butterfly needle mechanism is inserted in the sheath.

The patent aims at concealing and locking a needle in the sheath after its use. The arcuate mouth can facilitate the insertion of the wings in the sheath through the V-shaped groove. Further, the wings of the butterfly needle mechanism are locked and locked in the slot when the butterfly needle mechanism is inserted in the sheath. However, the patent suffered from several disadvantages. For example, the gaps may be excessively wide. Hence, the wings of the butterfly needle mechanism may disengage from the slot by leaving the slanted portions when an external force is applied to the butterfly needle mechanism. As a result, the needle is projected from a mouth of the sheath for causing a possibility of accidentally pricking a medical worker. Moreover, the patent only has a simple construction. This inhibits a further improvement thereof. Thus, it is desirable to provide a novel disposable butterfly needle sheath with highly safety characteristic in order to overcome the above drawbacks of the prior art.

### SUMMARY OF THE INVENTION

It is an object of the present invention to provide a butterfly needle sheath which, after use, a needle thereof can be concealed and locked prior to throwing away the needle sheath. By utilizing this, a possibility of accidentally pricking a medical worker after use is substantially eliminated.

In one aspect of the present invention a disposable needle sheath assembly comprises a butterfly needle mechanism comprising a needle having a rear end coupled to a plastic intravenous tubing and two wings at both sides of the needle; and a plastic sheath comprising a forward aperture, two rear side slits in communication with outside and inside of the sheath, two pairs of upper female snapping members each including a hole and lower male snapping members each including an upward peg in a rear end, each pair being separated by the slit, a latched member in forward ends of the slits, a slot extended forward from a top of the latched member to a predetermined position adjacent the aperture, the slot being in communication with the latched member; wherein prior to use the butterfly needle mechanism is inserted in the sheath with the needle concealed, the wings anchored on the slot, and the upward pegs are inserted into the holes for securing the upper female snapping members and the lower male snapping members together; in use push the wings forward along the slot until the needle projects from the aperture; and after use pull the wings rearward until the needle has completely concealed in the sheath and a joint of the wings has moved in the slits prior to pushing the wings forward again until being stopped by the latched member.

In another aspect of the present invention a disposable needle sheath assembly comprises a butterfly needle mechanism comprising a needle having a rear end coupled to a plastic intravenous tubing and two wings at both sides of the needle; and a plastic sheath comprising a forward aperture, a rear side slit in communication with outside and inside of the sheath, a pair of upper female snapping member including a hole and lower male snapping member including an upward peg in a rear end, the pair being separated by the slit, a latched member in a forward end of the slit, a slot extended forward from a top of the latched member to a predetermined position adjacent the aperture, the slot being in communication with the latched member; wherein prior to use the butterfly needle mechanism is inserted in the sheath with the needle concealed, the wings anchored on the slot, and the upward peg is inserted into the hole for securing the upper female snapping member and the lower male snapping member together; in use push the wings forward along the slot until the needle projects from the aperture; and after use pull the wings rearward until the needle has completely concealed in the sheath and a joint of the wings has moved in the slit prior to pushing the wings forward again until being stopped by the latched member.

The above and other objects, features and advantages of the present invention will become apparent from the following detailed description taken with the accompanying drawings.

### BRIEF DESCRIPTION OF THE DRAWINGS

FIG. 1 is an exploded perspective view of a first preferred embodiment of disposable butterfly needle sheath according to the invention;

FIG. 2 is a perspective view of the assembled butterfly needle sheath with a needle concealed therein;

FIG. 3 is a cross-sectional view of FIG. 2;

FIG. 4 is a view similar to FIG. 3 where the needle has projected from a sheath of the invention;

FIG. 5 is a perspective view of FIG. 4;

FIG. 6 is a view similar to FIG. 3 where the needle has concealed in the sheath but not locked;

FIG. 7 is a view similar to FIG. 3 where the needle has concealed and locked in the sheath;

FIG. 8 is a perspective view of FIG. 7;

FIG. 9 is a cross-sectional view of fastened two pairs of upper female snapping members and lower male snapping members;

FIG. 10 is a view similar to FIG. 9 where only one pair of upper female snapping member and lower male snapping member and a single slit are formed according to a second preferred embodiment of disposable butterfly needle sheath of the invention; and

FIG. 11 is a perspective view of a third embodiment of the invention.

### DETAILED DESCRIPTION OF THE PREFERRED EMBODIMENTS

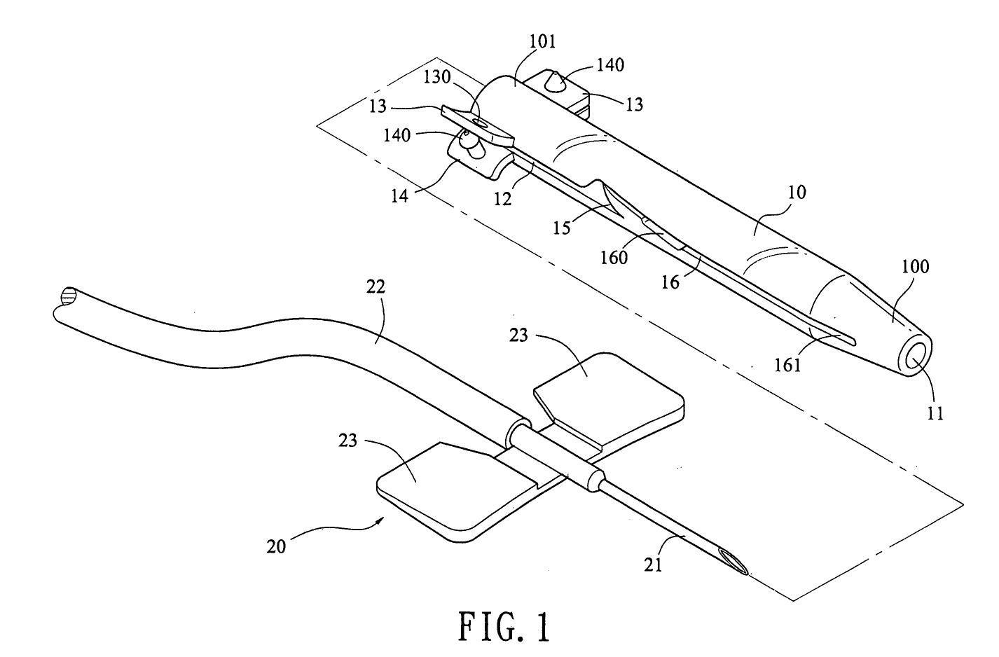

Referring to FIGS. 1, 2, 3, and 9, there is shown a disposable butterfly needle sheath constructed in accordance with the invention comprising a butterfly needle mechanism 20 and a plastic sheath 10. Each component is described in detail below.

The butterfly needle mechanism 20 comprises a needle 21 for introducing into the blood vessel, a plastic IV tubing 22 coupled to a rearward end of the needle 21, and two wings 23 at both sides of the needle 21. The sheath 10 comprises a bore 11, a tapered forward portion 100 for permitting the needle 21 to extend or retract through the bore 11 at the tapered forward portion 100, a rearward portion 101, two side slits 12 in the rearward portion 101 in communication with outside and the bore 11, and two pairs of upper female snapping members 13 and lower male snapping members 14 in a rear end, each pair being separated by the slit 12 in which the upper female snapping member 13 comprises a through hole 130 and the lower male snapping member 14 comprises an upward peg 140 respectively. The upward pegs 140 are inserted through the through holes 130 for securing the upper female snapping members 13 and the lower male snapping members 14 together after the butterfly needle mechanism 20 has inserted in the sheath 10 as detailed later. The sheath 10 further comprises a triangular latched member 15 in forward ends of the slits 12, a slanted slot 16 extended forward, obliquely from a top of the latched member 15 to a horizontal section 161 adjacent the forward portion 100 of the sheath 10 in which the slanted slot 16 is in communication with the latched member 15 and an intermediate part of the slanted slot 16 is formed as a recess 160 for permitting the wings 23 to anchor thereon.

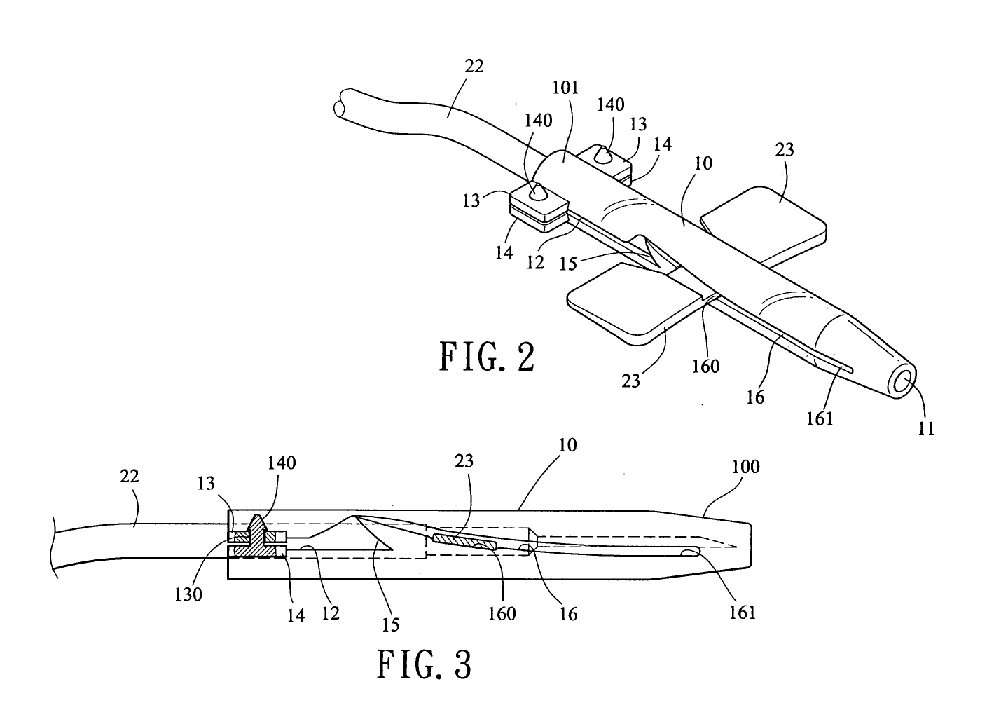

An assembly of the butterfly needle sheath will be described in detail below by referring to FIGS. 2 and 3 specifically. First, insert the butterfly needle mechanism 20 in the sheath 10 from the rearward portion 101 until the wings 23 are seated on the recess 160 by passing a rear section of the slanted slot 16. Next, insert the upward pegs 140 through the through holes 130 for securing the upper female snapping members 13 and the lower male snapping members 14 together. This finishes the assembly of the butterfly needle sheath.

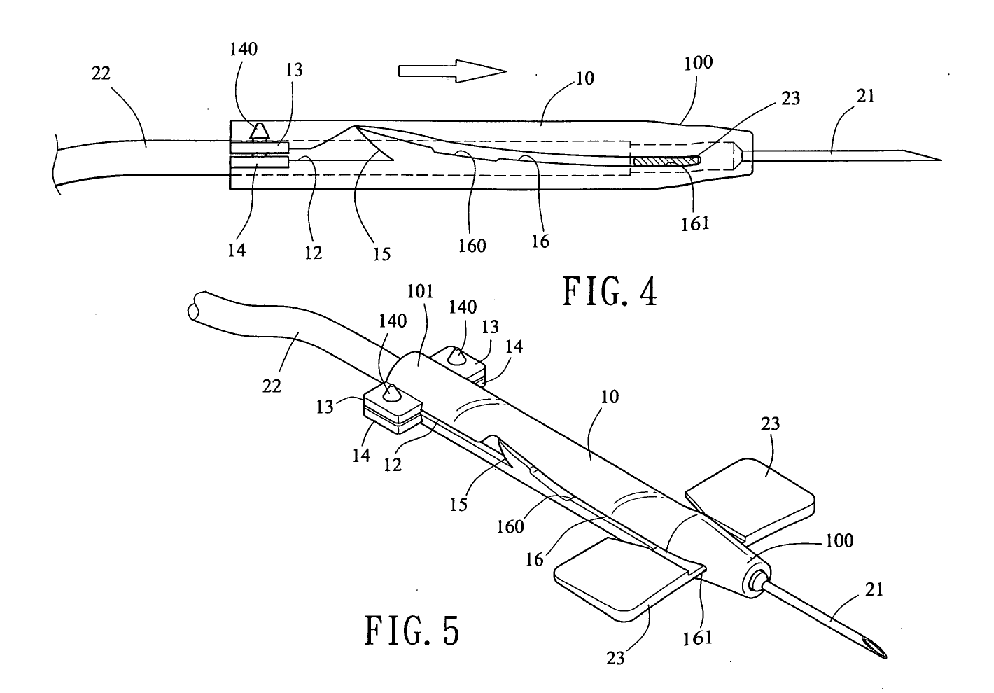

Referring to FIGS. 4 and 5, an operation of the butterfly needle sheath will be described in detail below. A user can push the wings 23 from the recess 160 toward the forward portion 100 along the a forward section of the slanted slot 16 until the needle 21 projects from the forward portion 100 by passing the horizontal section 161. Once the needle 21 is projected, a medical worker may introduce it into the blood vessel of a patient.

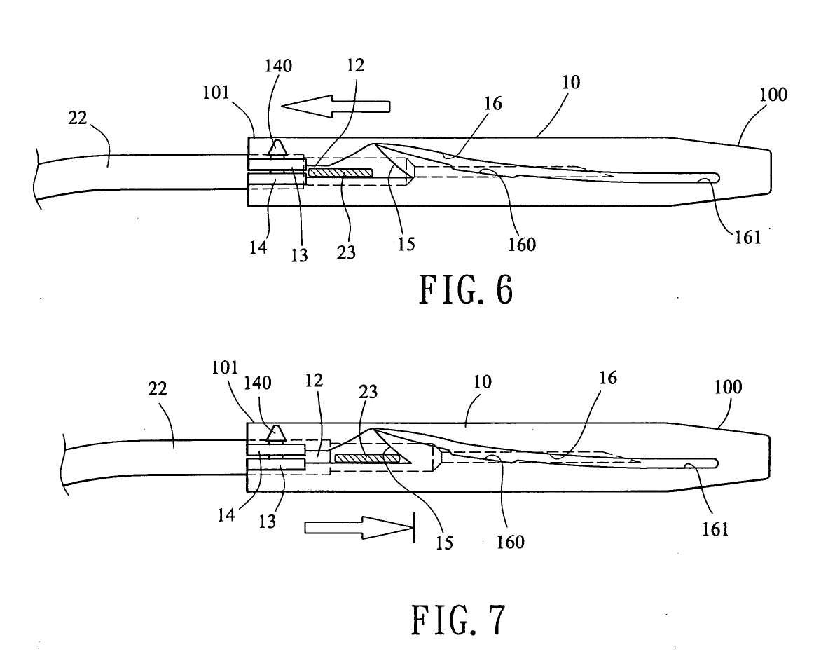

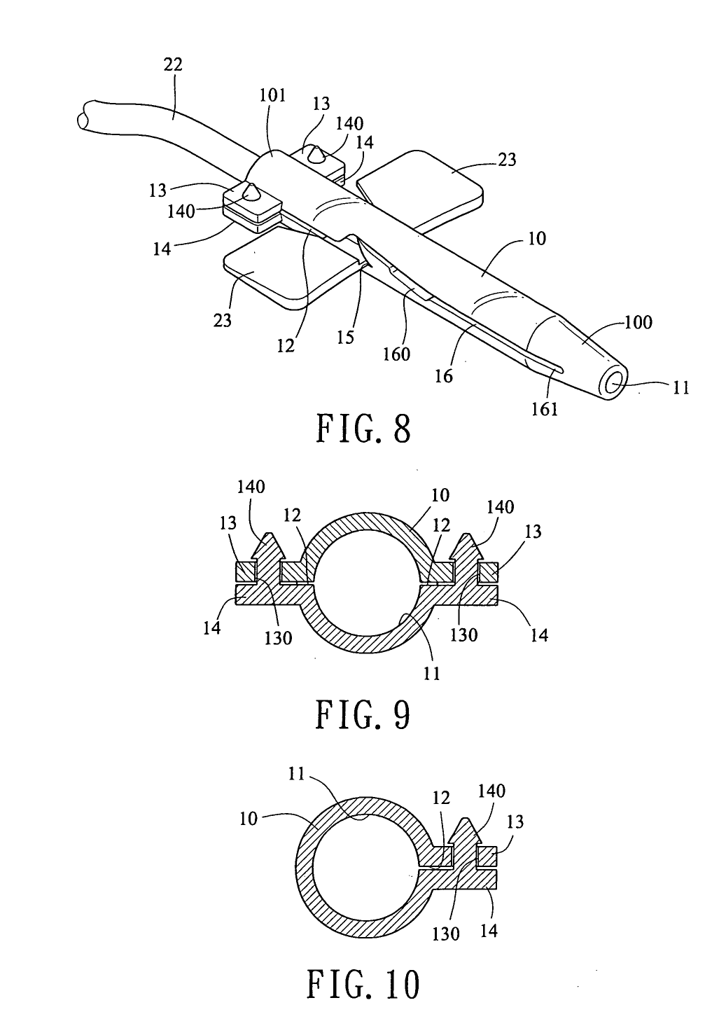

Referring to FIGS. 6, 7, and 8, an operation of throwing away the butterfly needle sheath after use will be described in detail below. First, a user can pull the wings 23 rearward toward the latched member 15 until the needle 21 has completely concealed in the bore 11 and a joint of the wings 23 has moved in the slits 12. At this position, the user has to push the wings 23 forward again until the wings 23 are stopped by the edge of the latched member 15 for locking. Note that the triangular latched member 15 aims at preventing the stopped wings 23 from moving toward the slanted slot 16 for projecting the needle 21 again once a forward pushing force is applied to the wings 23. As an end, a possibility of accidentally pricking a medical worker by the needle 21 after use is substantially eliminated. Another purpose of the triangular latched member 15 is to completely prevent criminals from selling the butterfly needle sheaths to hospitals again by simply cleaning or even disinfecting the used needles.

Further note that the latched member 15 may be shaped other than a triangle in any of other embodiments as long as it can lock or fasten the wings 23. Moreover, the latched member 15 can be formed only at one side of front ends of the slits 12. Also, a rear section of the slanted slot 16 can be formed as a flat section rather than extends forward, obliquely. In addition, the intermediate recess 160 may be shaped other than a recess.

Referring to FIG. 10, there is shown a second preferred embodiment of disposable butterfly needle sheath according to the invention in which only one pair of upper female snapping member 13 and lower male snapping member 14 and a single slit 12 are formed at one side of the sheath 10. This is different from the first preferred embodiment shown in FIG. 9.

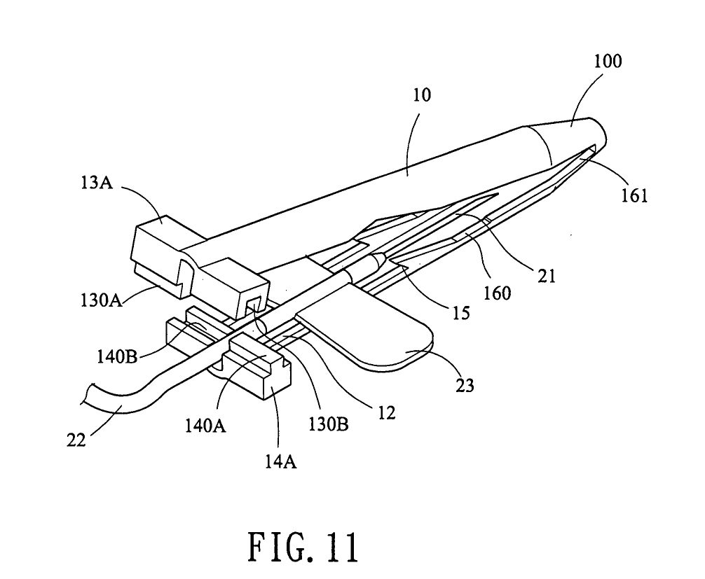

Regarding to FIG. 11, there is shown a third preferred embodiment of disposable butterfly needle sheath according to the invention wherein all of the constructions components are same as that of the first embodiment of the invention as described above, except the upper snapping member 13A and the lower snapping member 14A. As shown in FIG. 11, in this embodiment, the upper snapping member 13A has a downward projection 130A and a downward recess 130B, oppositely, the lower snapping member 14A has a upward projection 140A and a upward recess 140B. In operation, the downward projection 130A and downward recess 130B of the upper snapping member 13A are fastened to the upward recess 140B and the upward projection 140A of the lower snapping member 14A, respectively after having the butterfly needle mechanism 20 inserted in the sheath 10, so as to secure the upper snapping member 13A and the lower snapping member 14A together as one piece as mentioned above. The remaining components designated with same reference numbers serving as same function as above, the related descriptions are thus omitted.

While the invention herein disclosed has been described by means of specific embodiments, numerous modifications and variations could be made thereto by those skilled in the art without departing from the scope and spirit of the invention set forth in the claims.

What is claimed is:

1. A disposable butterfly needle sheath assembly comprising:
a butterfly needle mechanism comprising a needle having a rear end coupled to a plastic intravenous tubing and two wings at both sides of the needle; and
a plastic sheath comprising a forward aperture, two rear side slits in communication with outside and inside of the sheath, two pairs of upper female snapping members each having a hole and lower male snapping members each having an upward peg in a rear end, each pair being separated by the slit, a latched member in the slits, a slot extended forward from a top of the latched member to a predetermined position adjacent the aperture, the slot being in communication with the latched member;
wherein prior to use the butterfly needle mechanism is inserted in the sheath with the needle concealed, the wings anchored on the slot, and the upward pegs are inserted into the holes for securing the upper female snapping members and the lower male snapping members together; in use push the wings forward along the slot until the needle projects from the aperture; and after use pull the wings rearward until the needle has completely concealed in the sheath and a joint of the wings has moved in the slits prior to pushing the wings forward again until being stopped by the latched member.

2. The disposable needle sheath assembly of claim 1, wherein a forward portion of the slot is formed as a horizontal section.

3. The disposable needle sheath assembly of claim 1, wherein an intermediate portion of the slot is formed as a recess for anchoring the wings.

4. The disposable needle sheath assembly of claim 1, wherein the latched member is shaped as a triangle.

5. The disposable needle sheath assembly of claim 1, wherein a rearward portion of the slot extends forward, obliquely.

6. A disposable needle sheath assembly comprising:
a butterfly needle mechanism comprising a needle having a rear end coupled to a plastic intravenous tubing and two wings at both sides of the needle; and
a plastic sheath comprising a forward aperture, a rear side slit in communication with outside and inside of the sheath, a pair of upper female snapping member having a hole and lower male snapping member having an upward peg in a rear end, the pair being separated by the slit, a latched member in the slit, a slot extended forward from a top of the latched member to a predetermined position adjacent the aperture, the slot being in communication with the latched member;
wherein prior to use the butterfly needle mechanism is inserted in the sheath with the needle concealed, the wings anchored on the slot, and the upward peg is inserted into the hole for securing the upper female snapping member and the lower male snapping member together; in use push the wings forward along the slot until the needle projects from the aperture; and after use pull the wings rearward until the needle has completely concealed in the sheath and a joint of the wings has moved in the slit prior to pushing the wings forward again until being stopped by the latched member.

7. The disposable needle sheath assembly of claim 6, wherein a forward portion of the slot is formed as a horizontal section.

8. The disposable needle sheath assembly of claim 6, wherein an intermediate portion of the slot is formed as a recess for anchoring the wings.

9. The disposable needle sheath assembly of claim 6, wherein the latched member is shaped as a triangle.

10. The disposable needle sheath assembly of claim 6, wherein a rearward portion of the slot extends forward, obliquely.

11. A disposable needle sheath assembly comprising:
a butterfly needle mechanism comprising a needle having a rear end coupled to a plastic intravenous tubing and two wings at both sides of the needle; and
a plastic sheath comprising a forward aperture, a rear side slit in communication with outside and inside of the sheath, a pair of upper snapping member having a downward projection and a downward recess, and lower snapping member having an upper recess and an upper projection, the pair being separated by the slit, a latched member in the slit, a slot extended forward from a top of the latched member to a predetermined position adjacent the aperture, the slot being in communication with the latched member;
wherein prior to use the butterfly needle mechanism is inserted in the sheath with the needle concealed, the wings anchored on the slot, and the downward projection and the downward recess of the upper snapping member are coupled to the upward recess and the upward projection of the lower snapping member, respectively so as to secure the upper snapping member and the lower snapping member together; in use push the wings forward along the slot until the needle projects from the aperture; and after use pull the wings rearward until the needle has completely concealed in the sheath and a joint of the wings has moved in the slit prior to pushing the wings forward again until being stopped by the latched member.

12. The disposable needle sheath assembly of claim 11, wherein a forward portion of the slot is formed as a horizontal section.

13. The disposable needle sheath assembly of claim 11, wherein an intermediate portion of the slot is formed as a recess for anchoring the wings.

14. The disposable needle sheath assembly of claim 11, wherein the latched member is shaped as a triangle.

15. The disposable needle sheath assembly of claim 11, wherein a rearward portion of the slot extends forward, obliquely.
